(19) United States
(12) Patent Application Publication
(10) Pub. No.: US 2022/0234340 A1
(43) Pub. Date: Jul. 28, 2022

(54) MULTI-LAYER SELF-ADHESIVE SURFACE PROTECTION FILM AND METHODS OF USE

(71) Applicant: James Parson, Calgary (CA)

(72) Inventor: James Parson, Calgary (CA)

(21) Appl. No.: 17/248,526

(22) Filed: Jan. 28, 2021

(51) Int. Cl. B32B 27/20; B32B 7/06; B32B 7/12; B32B 25/20

(52) U.S. Cl. CPC B32B 27/20; B32B 7/06; B32B 7/12; B32B 25/20; B32B 2255/10; B32B 2405/00; B32B 2264/1051; B32B 2264/302; B32B 2264/301; B32B 2307/4026; B32B 2307/7145; B32B 2255/205

(57) ABSTRACT

Embodiments of the present invention relate to pressure-sensitive adhesive plastic film or sheet optionally having antimicrobial properties. The film or sheet can be applied to numerous frequent use objects and finds particular utility in the health care field. The pressure-sensitive adhesive plastic film or sheet can be supplied in pads that can be applied to both the outermost, or patient, side of the face shield, or it can be applied to the innermost, or healthcare professional, surface. After the object, such as a face shield, has been used, the top layer of the pressure-sensitive adhesive plastic can be removed and discarded.

### FIELD OF THE INVENTION

The present disclosure relates to multi-layer protective film or sheet, particularly to multi-layer self-adhesive protective films or sheets, more particularly to the use of a multi-layer self-adhesive medical grade protective films in various consumer products and medical personal protective equipment (PPE) applications.

### BACKGROUND

US20100104791 discloses a method for the antimicrobial protection of an object using an antimicrobial pressure-sensitive adhesive plastic film. Due to its self-adhesive nature, the film and thus the surface antimicrobial protection, can easily be applied by unqualified personnel or by a person without specific equipment. Similarly, the film can be removed as easily as it was applied and then replaced, again without specific equipment,

WO2013011656 discloses a pressure sensitive adhesive film or sheet, surface protective film or sheet, and method used to protect the surface of an article. The adhesive film or sheet is used as a surface protective film for protecting the surface of a product from dust adhesion, dirt, scratches, and the like. The pressure-sensitive adhesive film or sheet in the present embodiment is attached only by pressure so that the adhesive layer faces the surface of a hard adherend. When not needed, it is easily peeled off by hand, leaving no contaminants on the surface of the adherend.

U.S. Pat. No. 9,247,736 discloses antimicrobial adhesive films. A multi-layer film for reducing microbial contamination on a surface. The multi-layer film can include a core layer having a first surface and a second surface opposite the first surface, an adhesive layer disposed adjacent the first surface of the core layer, and an antimicrobial layer disposed adjacent the second surface. The antimicrobial adhesive films are for use on surfaces to reduce the risk of contamination by undesired microorganisms. The multi-layer film is adhered to surface and has an antimicrobial adhesive film that includes antimicrobial hardcoat layer, core layer, and adhesive layer.

U.S. Pat. No. 3,600,268 discloses a surface protecting sheet. The surface protecting sheet comprises a flexible plastic sheet of polyvinyl chloride, etc. It has a thickness of 0.20 to 0.02 mm. And a cured acrylic rubber layer not over 0.05 mm. Thick on one surface thereof, said rubber layer being made by cross-linking a synthetic rubber consisting mainly of acrylic ester. A layer of low frictional resistance is preferably provided on the other surface of the flexible plastic sheet. The sheet is useful in protecting the surface of a plate or sheet, while affording a view of such surface. The present sheet has high resistance to light and weather, does not tend to adhere to the protected surface when removed therefrom, and is sufficiently strong to resist pressures applied.

U.S. Pat. No. 5,895,714 discloses a self-adhesive protective film. The protective film comprises a self-adhesive protective film with a film backing coated with a self-adhesive composition of polyethylene vinyl acetate. The self-adhesive protective film, is especially suitable for cars.

U.S. Pat. No. 9,127,189 discloses a coating for objects, in particular in public facilities and/or means of transportation, for preventing the transmission of infections. The coating is intended to be applied externally to surfaces of objects. The covering material is flexible and can be adapted to the outer shape of the object. An adhesive bonding may be used for connecting the covering to the object. For this purpose, the covering may be accompanied, for example in the form of a set, by an adhesive, which is applied to the object before the covering is applied

US20060082085 discloses an improved sanitary sheath for a cart handle. A disposable sanitary sheath for a shopping cart handle featuring a sheet wrappable about said handle, said sheet having a length and width so as to cover at least the portion of said handle coming into contact with a user's hands, an adhesive strip positioned about one side of a surface of said sheet for securing said sheet about said handle, a perforated pull-away strip disposed along the length of said sheet, and a pull tab accommodated on said pull-away strip. Other embodiments include disposable sanitary sheaths for shopping cart handles with a sheet wrappable about said handle. Additionally, a sanitizing compound such as an antibacterial agent can be applied to the sheath.

DE4311322 discloses multi-layer, self-adhesive surface protection film. A self-adhesive multi-layer protection film based on polyolefins comprises: a carrier layer having a layer thickness in the range of from 20 to 150 mu m and comprising one or more layers of polyolefins; and an adhesive layer having a layer thickness in the range of from 5 to 40 mu m and comprising 99.5-80 wt. % of an ethylene vinyl ester copolymer and/or polyethylene or a mixture thereof; and from 0.5 to 20 wt. % of one or more adhesion-promoting resins. Such a film is of particular use in protection of the surfaces of plastics sheets during thermal forming, storage and transportation.

US20120251756 discloses adhesive backed antimicrobial cover for use on high volume touch surfaces. A cover member is provided for attachment to a contacted surface having a predetermined size and shape. The contacted surface is a surface that is regularly subjected to physical contact. The cover member includes a sheet-like substrate and an adhesive layer is disposed on the surface of the substrate. A releasable backing member is attached to the adhesive layer, and an antimicrobial coating is disposed on the first surface of the substrate. The present invention relates to products for covering surfaces, and more particularly, to an adhesive backed cover device that include an antimicrobial surface. The cover of the present invention has special utility on high touch surfaces that are touched by a large number of people, such as elevator buttons and touch screens.

CN105966024 discloses a high transparency type self-adhesive surface protection film and manufacturing method thereof. The high transparency type self-adhesive surface protection film comprises a self-adhesive layer, an intermediate layer and an anti-sticking layer, wherein the self-adhesive layer comprises ultra low density polyethylene and flexible polypropylene; the intermediate layer is prepared by high-transparency copolymerized polypropylene, and the anti-sticking layer is prepared by a ethylene-propylene random copolymer. Also disclosed is a method for manufacturing the high transparency type self-adhesive surface protection film. The film can be widely applied to surface protection fields of transparent optical sheet materials, high gloss paint surface, and other products.

US20050129937 discloses and antimicrobial web for application to a surface. The web has a flexible support layer having a first side and a second side, a flexible antimicrobial layer adjacent the first side of the support layer, and a flexible adhesive layer adjacent the second side of the support layer. The medium containing a controlled release antimicrobial material and/or that changes visual appearance as the material reaches a predetermined state. The medium also has an adhesive layer so it can be adhered to a surface such as a counter top.

U.S. Pat. No. 8,176,685 discloses protective sheet made from a blend of ethylene vinyl acetate and polyethylene. The protective sheet or drop cloth is made from a blend comprising ethylene vinyl acetate (EVA) and polyethylene (PE), making it naturally liquid impervious and slip resistant. The protective sheet includes a top side having a raised surface design configured for retaining fluid, and a bottom side that is smoother than the top side for added slip resistance. The method comprises the steps of providing the protective sheet and positioning it over the object, wherein the top side faces away from the object, and the bottom side faces the object and the top side is available for a workman to walk on.

None of the above cited documents, alone or in combination, satisfy the need for multi-layer protective films, particularly to multi-layer self-adhesive protective films. There therefore remains a need for multi-layer self-adhesive medical grade protective films in various consumer products applications.

### BRIEF SUMMARY

It is an object of the invention to provide a multi-layer self-adhesive surface protection film and methods of use.

In accordance with an aspect of the invention there is provided a self-adhesive protection film, comprising a film fabricated from a medical grade plastic, said film having an upper surface and a lower surface; a pressure sensitive adhesive coated on to said lower surface of said film; and a tab attached to an outer edge of said film, wherein said film is sized and proportioned to be applied to an outer patient facing or inner health care professional surface of a personal protective equipment face shield.

In accordance with another aspect of the invention there is provided a pad of self-adhesive protection films, comprising a plurality of films fabricated from a medical grade plastic, said films having an upper surface and a lower surface; a pressure sensitive adhesive coated on to said lower surface of said films; and tab attached to an outer edge of said films, wherein said film is sized and proportioned to be applied to a multitude of frequent use objects.

In accordance with yet another aspect of the invention there is provided a pad of self-adhesive protection films, comprising a plurality of plastic films having an upper surface and a lower surface; a pressure sensitive adhesive coated on to said lower surface of said films; and a tab attached to an outer edge of said films, wherein said film is sized and proportioned to be applied to a motor vehicles or helmet visors to protect from dirt or damage.

The advantages and features of the present invention will become better understood with reference to the following more detailed description and claims taken in conjunction with the accompanying drawings in which like elements are identified with like symbols.

To easily identify the discussion of any particular element or act, the most significant digit or digits in a reference number refer to the figure number in which that element is first introduced.

### BRIEF DESCRIPTION OF THE DRAWINGS

To easily identify the discussion of any particular element or act, the most significant digit or digits in a reference number refer to the figure number in which that element is first introduced.

FIG. 1 illustrates an aspect of the subject matter in accordance with one embodiment of the invention.

FIG. 2 illustrates front and perspective views of an aspect of the subject matter in accordance with one embodiment of the invention.

FIG. 3 illustrates perspective views of aspects of the subject matter in accordance with one embodiment of the invention.

FIG. 4 illustrates a perspective view of an aspect of the subject matter in accordance with one embodiment of the invention.

FIG. 5 illustrates perspective views of aspects of the subject matter in accordance with further embodiments of the invention.

FIG. 6 illustrates perspective views of aspects of the subject matter in accordance with further embodiments of the invention.

FIG. 7 illustrates various aspects of the subject matter in accordance with multiple embodiments of the invention.

### DETAILED DESCRIPTION

Devices and methods for carrying out the invention are presented in terms of embodiments depicted within the FIGS. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

Embodiments of the present invention relate to pressure-sensitive adhesive plastic film or sheet optionally having antimicrobial properties.

Due to its self-adhesive nature, the film and thus the surface antimicrobial protection, can easily be applied by unqualified personnel or by a person without specific equipment. Similarly, the film can be removed as easily as it was applied and then be safely disposed of without specific equipment,

In the embodiments having antimicrobial properties, the film that is applied to surfaces renders the outer surface of the object active against microbes, including bacteria, viruses, fungi, and algae, so as to prevent the propagation of the microbes.

The antimicrobial properties may be provided by silver nanoparticles applied to the surface of the pressure-sensitive adhesive plastic film or sheet, or it may be present within the adhesive layer.

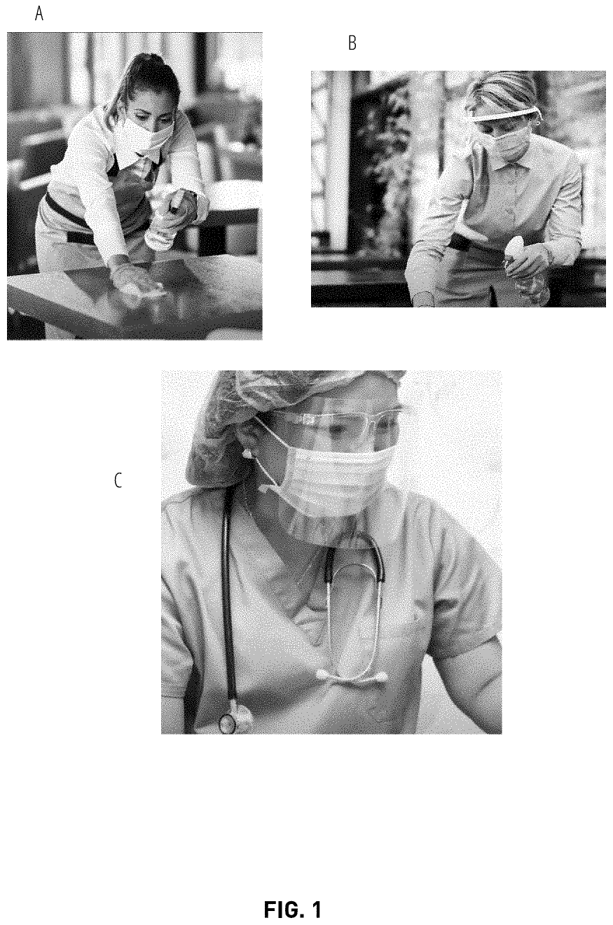

FIG. 1 illustrates the need for various forms of PPE brought on by the recent COVID-19 pandemic. FIG. 1A shows a hospitality worker sanitizing a table surface wearing a disposable mask. FIG. 1B shows a safer form of PPE for the hospitality worker, namely a plastic face shield offering additional protection above and beyond the disposable mask. FIG. 1C shows a health can worker wearing both a disposable mask and a plastic face shield.

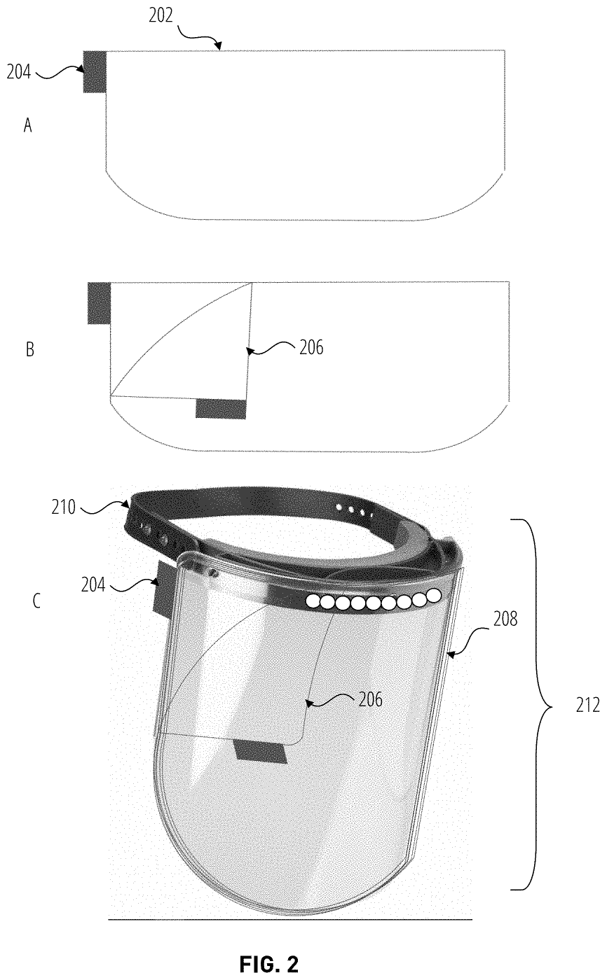

FIG. 2A-C illustrate more detailed views of a plastic face shield and proposed inventive attachments. FIGS. 2A and B show front views of a pad of plastic visor covers 202, each plastic visor cover 202 having an outer tab 204. It can be seen that the individual plastic visor covers 202 can be removed, one cover at a time, by pulling on the outermost outer tab 204.

A perspective view (C) of a standard face shield can be seen having numerous plastic visor covers 208 affixes to the outer surface of the face shield.

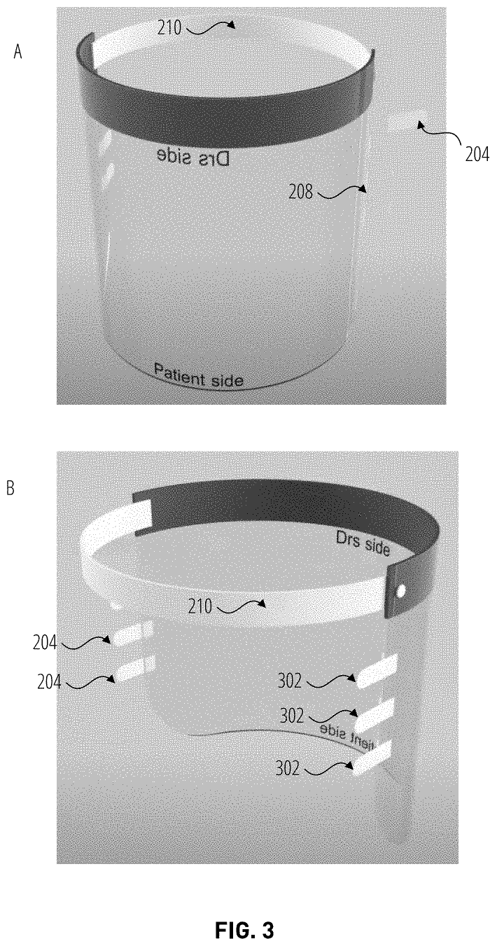

FIGS. 3A and B illustrate additional embodiments of the invention showing application of the pressure-sensitive adhesive plastic film or sheet affixed to both the inner (doctor) surface of the shield and the outer (patient) surface of the shield. This application of the film or sheet to both sides of the plastic face shield allows for continued use as opposed to the current practice of discarding or re-sterilizing masks during the work day.

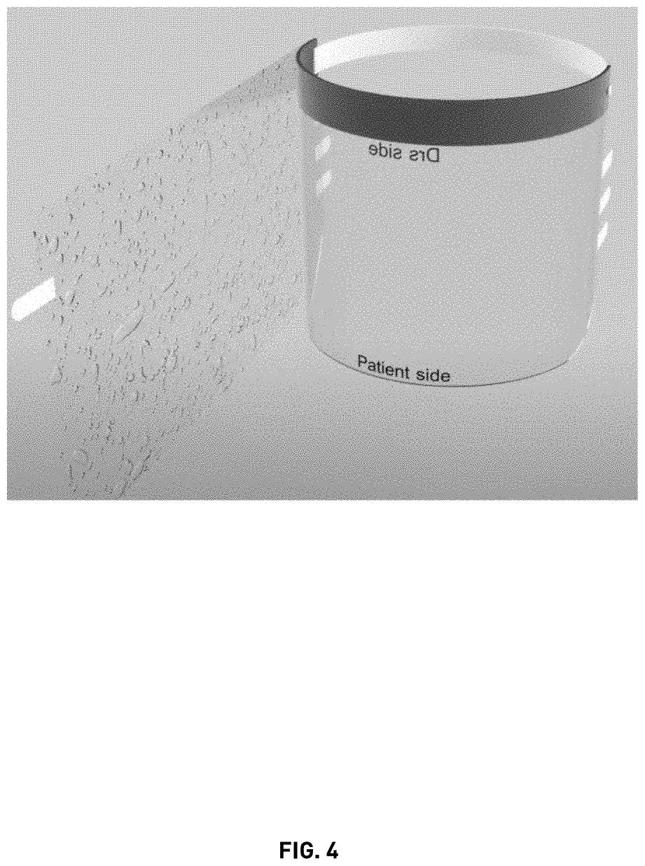

FIG. 4 illustrates the removal of the outermost plastic visor cover 202 on the patient side of a plastic face shield 212 or visor. This is achieved by pulling on the outermost outer tab 204 on the patient side of the shield or visor.

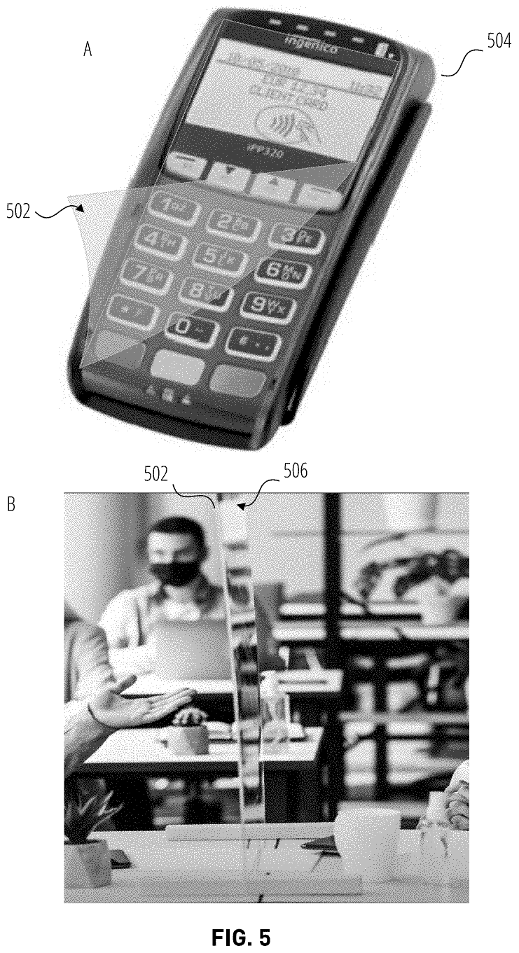

FIGS. 5A and B illustrates additional embodiments of the invention when used on ATM machines and Perspex™ dividers.

As can be seen in FIG. 5A the removable pressure-sensitive adhesive plastic film 502 can be used to protect an ATM terminal 504 or similar pay, or selection, stations at various points of sale, including but not limited to shops, gas station pumps, car wash terminals, electric vehicle charging points, and the like.

FIG. 5B shows the use of the removable pressure-sensitive adhesive plastic film 502 applied to a Perspex divider 506. Perspex dividers 506 are more and more becoming common place in situations where it is necessary for people to interact at distances within the current social distancing requirements. The Perspex dividers 506 can be seen in most shops, situated between the cashier and the customer. They can also be found separating desk or work spaces in schools, universities, colleges, cubicles in offices, between or on tables in restaurants, on counters in all government offices, and the like. In this particular embodiment, it is contemplated that a removable pressure-sensitive adhesive plastic film 502 can be applied to both sides of the Perspex divider 506, thereby protecting subsequent customers and alternate employees at shift change from contamination with microbes or microorganisms including, but not limited to bacteria, viruses, algae, and fungi.

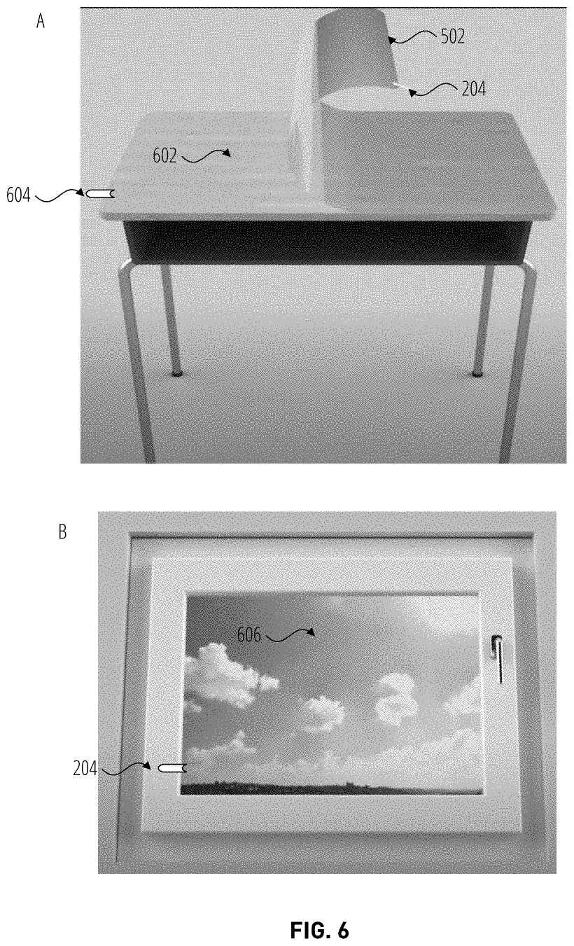

FIGS. 6A and B illustrate yet additional uses of embodiments of the removable pressure-sensitive adhesive plastic films 502 of the present invention.

FIG. 6A shows the removal of the upper sheet of the removable pressure-sensitive adhesive plastic film 502. In this embodiment the sheet or film is applied to a table top or desk. The sheet being removed by pulling the outer tab 204 which lifts the uppermost sheet and allows for easy removal and subsequent disposal. All number of different table tops, work surfaces or desks can be covered with the sheet or film, particularly in the travel industry, such as foldaway table tops in airplanes.

FIG. 6B shows a further use of the removable pressure-sensitive adhesive plastic film 502 as applied to a window pane 606. It is contemplated that the film or sheet can be applied to windows or mirrors in all manner of different public spaces, including but not limited to shops, restaurants, washrooms, airplanes, schools, offices and the like. The film can be applied to both sides of the window pane 606.

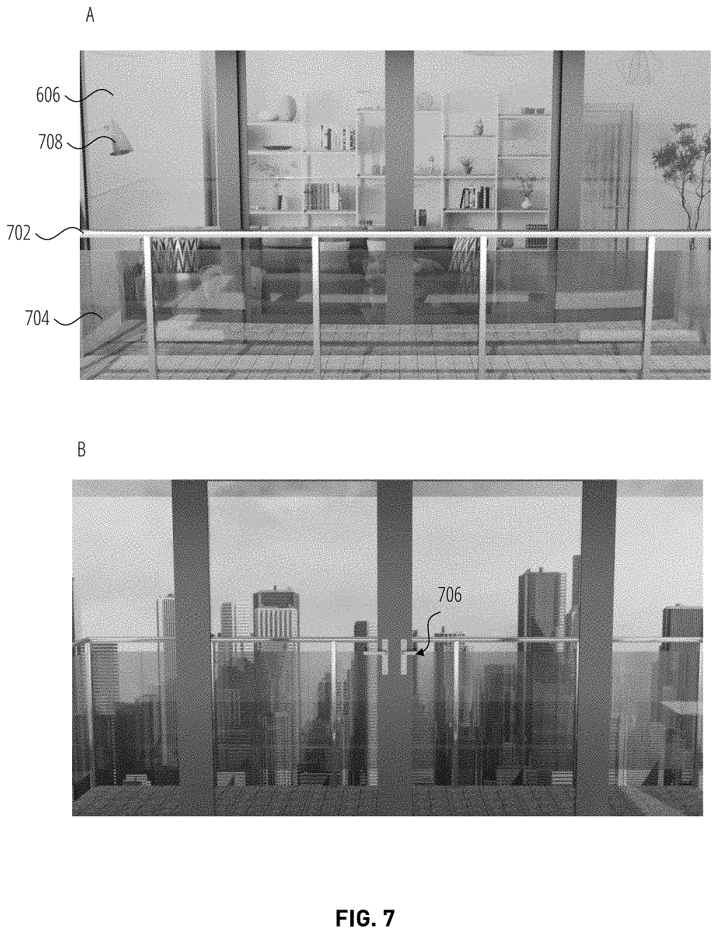

FIGS. 7A and B illustrate numerous different potential uses of the removable pressure-sensitive adhesive plastic film 502 of embodiments of the invention.

The removable pressure-sensitive adhesive plastic film 502 can be applied to railings 702, window panes 606, safety screens 704, door handles 706, and many indoor surfaces such as lamps 708.

In conclusion, it can be seen that there are many different potential used for the removable pressure-sensitive adhesive plastic film 502.

Some uses may be specifically suitable to provide sanitary protection from microbes or microorganisms including, but not limited to bacteria, viruses and fungi. In such instances, it is contemplated that the removable pressure-sensitive adhesive plastic film 502 is fabricated from medical grade plastics. Medical grade plastic materials are an essential part of the modern healthcare system. These materials comply with global regulatory requirements and are specifically engineered and manufactured for medical use.

The following list includes FDA compliant plastics that have all the manufacturing advantages of plastic, but also contain properties that make them compatible for medical uses. The list of FDA approved plastics include: acrylonitrile butadiene styrene (ABS); acetal copolymer; acetal homopolymer; polyethylene terphthalate polyester; polyethylene-chlorotrifluoro-ethylene; polybutylene terephthalate-polyester; polyvinylidene fluoride; polyphenylene oxide; Nylon™; polyetheretherketone; polycarbonate; polyethylenes; polypropylene homopolymer; polyphenylsulfone; polysulfone; polyethersulfone; polyarylethersulfone; and Rulon™ 641. The plastic film of embodiments of the invention may be made from any, or a combination of, the above mentioned materials.

ABS plastic sheet is amongst the most versatile and widely used of thermoplastics, particularly within the thermoforming industry. The wide forming temperature range of ABS plastic sheet makes it relatively easy to form.

Pressure Sensitive Adhesives suitable for use in embodiments of the present invention typically include an elastomer which functions as the primary base material, which can be any one of the following materials: natural rubber; vinyl ethers; acrylics; butyl rubber; styrene block copolymers; silicones and nitriles. The adhesive of embodiments of the invention may be made from any, or a combination of, the above mentioned materials.

The removable pressure-sensitive adhesive plastic film 502 can be clear or tinted. The shade and intensity of tint can be varied to suit the end-users preference.

Typically, the self-adhesive protection films are formed into a pad. The pad comprising a plurality of films that are stacked laminarly with the outer surface of one film in contact with the inner surface of another film. The bottom film is in contact with a silicone release layer. A pressure sensitive adhesive coated on to a lower surface of the films, and a tab attached to an outer edge of each films. The pad of films is sized and proportioned to be applied to a multitude of frequent use objects.

Also included are methods of applying one or more pads of the self-adhesive protection films to objects such as, but not limited to, the inner and/or outer surfaces of the personal protective equipment face shield. The methods comprising the steps of: cleaning the surface to be protected, such as the inner and/or outer surfaces of the personal protective equipment face shield. Removing the silicone release layer from the bottom film of one pad and aligning one of the pads with the surface to be protected, such as the inner or outer surface of the personal protective equipment face shield. The self-adhesive film pad is then secured to the surface by applying gentle pressure, thereby affixing the adhesive layer to the surface to be protected.

When the object that has the protection film applied to it has been used, the outermost tab is pulled and the outermost film is removed and discarded.

The removable pressure-sensitive adhesive plastic films 502 can protect end users against cross-contamination or infection by way of the uses described herein. For example, pads of peelable removable pressure-sensitive adhesive plastic films 502 can be supplied in various different shapes and sizes for various applications. When the base sheet or film of the pad is affixed to the object to be covered, the end user is protected from contamination by frequent removal of the outer, potentially contaminated, removable pressure-sensitive adhesive plastic film 502.

In an alternative embodiment, additional microbial protection can be afforded by the incorporation of silver nanoparticles into the film or sheet, or onto the surface of the film or sheet. Nanoparticles of silver of between 1 nm and 100 nm in size. While frequently described as being ‘silver’ some are composed of a large percentage of silver oxide due to their large ratio of surface to bulk silver atoms.

Silver nanoparticles can prevent bacteria from growing on or adhering to the surface. This can be especially useful in surgical settings where all surfaces in contact with the patient must be sterile. Silver nanoparticles can be incorporated on many types of surfaces including metals, plastic, and glass.^{[85]} In medical equipment, it has been shown that silver nanoparticles lower the bacterial count on devices used compared to old techniques.

The ability to silver nanoparticles to prevent the spread of infection is due to release of silver (Ag⁺) ions from the surface of this material that is capable of destroying compounds that contain sulfur and phosphorus, such as the DNA and proteins present within bacteria, fungi, algae, or viruses.

In yet an alternative embodiment, the pressure sensitive adhesive may contain anti-microbial agents, such as antibacterial agents, antiviral agents, antialgal agents, and antifungal agents.

It is contemplated that the removable pressure-sensitive adhesive plastic film 502 can be applied to a variety of different frequent use objects selected from, but not limited to, the following: school desks; keyboards; computer mouse; touch screens, windows, piano keys; guitar necks at music stores; vehicle windows and mirrors; counter tops; bar counter tops; rides at fairs; glass doors; dental equipment such as lights; chalk boards; head light covers; LED light covers; taxi cab covers; sports helmets; Uber covers; heavy equipment covers; hand rails; elevator buttons; airplane tray covers; airplane windows; limo windows; dining tables; surfaces in rental vehicles; motorcycle or ATV quad windshields or fairings; motorcycle helmet visors; board room tables; restaurant tables; dividers and rear door windows in police cars, taxis and Ubers; city, school and charter bus windows, seats, rails and dividers; rental vehicle windows, condiments in restaurants; paper towel dispensers, and frequent use items in washrooms.

It is also contemplated that the removable pressure-sensitive adhesive plastic film 502 can be used as hospital bed wraps and hospital wheel chair wraps. The wraps can be made into car seat covers for rental companies or multi driver fleet trucks and vehicles or for stadium seating or theatres. All types of PPE masks, including athletic masks can use the film or sheet as described herein. As it can be used on the outside of all different types of mask to reduce transfer of pathogens, it can be applied to the inside or outside and be peeled off as and when required. For example, the outer surfaces of items left in a locker room or gym bag for any length of time, removal of a sheet or film would reduce the risk of infection.

An alternative use for the removable pressure-sensitive adhesive plastic films 502 is to provide physical protection of a variety of surfaces. In this form of use, the film or sheet may still be tinted or clear, but it does not need to be medical grade. For example, the custom cut pad of sheets or films could be sized and proportioned to fit onto vehicle windscreens, motorcycle fairings, motor racing helmet visors, and the like. In these embodiments, it is envisaged that the outer sheet or film can be torn from the pad to remove any dirt or insect impacts. The sheets or films can also offer protection from sandblasting damage in windy, arid locations.

The foregoing descriptions of specific embodiments of the present invention have been presented for purposes of illustration and description. They are not intended to be exhaustive or to limit the invention and method of use to the precise forms disclosed. Obviously many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

1. A self-adhesive protection film, comprising
a film fabricated from a medical grade plastic, said film having an upper surface and a lower surface;
a pressure sensitive adhesive coated on to said lower surface of said film; and
a tab attached to an outer edge of said film,
wherein said film is sized and proportioned to be applied to an outer patient facing or inner health care professional surface of a personal protective equipment face shield.

2. The self-adhesive protection film of claim 1, wherein said medical grade plastic is selected from the group consisting of: acrylonitrile butadiene styrene (ABS); acetal copolymer; acetal homopolymer; polyethylene terphthalate polyester; polyethylene-chlorotrifluoro-ethylene; polybutylene terephthalate-polyester; polyvinylidene fluoride; polyphenylene oxide; Nylon™; polyetheretherketone; polycarbonate; polyethylenes; polypropylene homopolymer; polyphenylsulfone; polysulfone; polyethersulfone; polyarylethersulfone; and Rulon™ 641; and combinations thereof.

3. The self-adhesive protection film of claim 1, wherein said pressure sensitive adhesive comprises an elastomer selected from the group consisting of: natural rubber; vinyl ethers; acrylics; butyl rubber; styrene block copolymers; silicones and nitriles; and combinations thereof.

4. The self-adhesive protection film of claim 1, wherein said pressure sensitive adhesive additionally comprises one of more agents selected from: antibacterial agents; antiviral agents; antialgal agents; and antifungal agents; and combinations thereof.

5. The self-adhesive protection film of claim 1, wherein silver nanoparticles are incorporated into said medical grade plastic.

6. The self-adhesive protection film of claim 1, wherein silver nanoparticles are coated onto said upper surface of said film.

7. The self-adhesive protection film of claim 1, wherein said film is tinted.

8. A pad made up of a plurality of said self-adhesive protection films of claim 1, said pad having a top film and a bottom film.

9. The pad of a plurality of said self-adhesive protection films of claim 8, wherein each of said plurality of films are stacked laminarly with said outer surface of one film in contact with said inner surface of another film, and said bottom film is in contact with a silicone release layer.

10. A method of applying one or more pads of said self-adhesive protection films of claim 9, to the inner and/or outer surfaces of said personal protective equipment face shield, comprising the steps of:
cleaning the inner and/or outer surfaces of said personal protective equipment face shield;
removing the silicone release layer from said bottom film of one pad;
aligning said one pad with the inner or outer surface of the personal protective equipment face shield and securing said one pad to the shield by applying gentle pressure; and optionally
removing the silicone release layer from said bottom film of another pad;
aligning said another pad with the outer or inner surface of the personal protective equipment face shield and securing said another pad to the shield by applying gentle pressure.

11. A method of claim 10, comprising the additional step of putting on said tab to remove said top film, on either the inner and/or outer surfaces of said personal protective equipment face shield after the face shield has been used.

12. A pad of self-adhesive protection films, comprising
a plurality of films fabricated from a medical grade plastic, said films having an upper surface and a lower surface;
a pressure sensitive adhesive coated on to said lower surface of said films; and
a tab attached to an outer edge of said films,
wherein said film is sized and proportioned to be applied to a multitude of frequent use objects.

13. The pad of self-adhesive protection films claim 12, wherein said medical grade plastic is selected from the group consisting of: acrylonitrile butadiene styrene (ABS); acetal copolymer; acetal homopolymer; polyethylene terphthalate polyester; polyethylene-chlorotrifluoro-ethylene; polybutylene terephthalate-polyester; polyvinylidene fluoride; polyphenylene oxide; Nylon™; polyetheretherketone; polycarbonate; polyethylenes; polypropylene homopolymer; polyphenylsulfone; polysulfone; polyethersulfone; polyarylethersulfone; and Rulon™ 641; and combinations thereof.

14. The pad of self-adhesive protection films claim 12, wherein said pressure sensitive adhesive comprises an elastomer selected from the group consisting of: natural rubber; vinyl ethers; acrylics; butyl rubber; styrene block copolymers; silicones and nitriles; and combinations thereof.

15. The pad of self-adhesive protection films claim 12, wherein said pressure sensitive adhesive additionally comprises one of more agents selected from: antibacterial agents, antiviral agents, antialgal agents, and antifungal agents.

16. The pad of self-adhesive protection films claim 12, wherein silver nanoparticles are incorporated into said medical grade plastic.

17. The pad of self-adhesive protection films claim 12, wherein silver nanoparticles are coated onto said upper surface of said film.

18. The self-adhesive protection film of claim 12, wherein said film is tinted.

19. The pad of self-adhesive protection films claim 12, wherein said frequent use objects are selected from the group consisting of: school desks; keyboards; computer mouse; touch screens, windows, piano keys; guitar necks at music stores; vehicle windows and mirrors; counter tops; bar counter tops; rides at fairs; glass doors; dental equipment such as lights; chalk boards; head light covers; LED light covers; taxi cab covers; sports helmets; Uber covers; heavy equipment covers; hand rails; elevator buttons; airplane tray covers; airplane windows; limo windows; dining tables; surfaces in rental vehicles; motorcycle or ATV quad windshields or fairings; motorcycle helmet visors; board room tables; restaurant tables; dividers and rear door windows in police cars, taxis and Ubers; city, school and charter bus windows, seats, rails and dividers; rental vehicle windows; condiments in restaurants; paper towel dispensers; hospital bed wraps; hospital wheel chair wraps; seat covers for rental vehicles, fleet trucks, stadium seating or theatre seating.

20. A pad of self-adhesive protection films, comprising
a plurality of plastic films having an upper surface and a lower surface;
a pressure sensitive adhesive coated on to said lower surface of said films; and
a tab attached to an outer edge of said films,
wherein said film is sized and proportioned to be applied to a motor vehicle windscreens or mirrors, or helmet visors, to protect from dirt or damage.
